(19) United States
(12) Patent Application Publication
(10) Pub. No.: US 2022/0160286 A1
(43) Pub. Date: May 26, 2022

(54) Adaptive User Interaction Systems For Interfacing With Cognitive Processes

(71) Applicants: Anhui Huami Health Technology Co., Ltd., Hefei (CN); Zepp, Inc., Cupertino, CA (US)

(72) Inventors: Aiden Arnold, Comox (CA); Yan Vule, Port Moody (CA); Artem Galeev, Vancouver (CA); Kongqiao Wang, Hefei (CN)

(21) Appl. No.: 17/504,056

(22) Filed: Oct. 18, 2021

Related U.S. Application Data
(60) Provisional application No. 63/116,292, filed on Nov. 20, 2020.

(51) Int. Cl. A61B 5/375; A61B 5/00

(52) U.S. Cl. CPC A61B 5/375; A61B 5/7264; A61B 5/7405; A61B 5/7455; A61B 5/681

(57) ABSTRACT

A method for modifying cognitive processes includes receiving respective electroencephalogram (EEG) signals from EEG sensors, where the EEG signals are of a brain of a user. Features are extracted from the respective EEG signals. A cognitive state of the brain of the user is obtained from a first machine learning (ML) model that uses the features as input. Feedback parameters of a feedback signal are obtained from a second model that uses the cognitive state as input. The feedback signal is and provided to the user and using a user device according to the feedback parameters.

### CROSS REFERENCES TO RELATED APPLICATION(S)

This disclosure claims the benefit of U.S. Provisional Application No. 63/116,292, filed Nov. 20, 2020, the disclosure of which is hereby incorporated by reference in its entirety.

### TECHNICAL FIELD

This disclosure generally relates to brain-computer interface (BCI), brain signal collection, and cognitive processes, and more specifically to modifying cognitive processes via adaptive feedback signals.

### BACKGROUND

Humans display numerous cognitive processes that allow them to function in physical and social environments. Cognitive processes have their origin in the brain. A cognitive process can be defined as the transmission of functionally relevant information within the neural systems of the brain, which correlates with brain activities (e.g., the brain dynamics) underlying (e.g., constituting, etc.) the cognitive process. Examples of cognitive processes include, but are not limited to, memory, attention, sensation, perception, thought, language, abstract representation, motor control, and so on.

The brain generates signals such as brain waves that can be measured, such as by an electroencephalogram (EEG). Changing a brain state or cognitive process can be effected in a change to generated brain waves.

### SUMMARY

A first aspect is a method for modifying cognitive processes. The method includes receiving respective electroencephalogram (EEG) signals from EEG sensors, where the EEG signals are of a brain of a user; extracting features from the respective EEG signals; obtaining, from a first machine learning (ML) model that uses the features as input, a cognitive state of the brain of the user; obtaining, from a second model that uses the cognitive state as input, feedback parameters of a feedback signal; and providing, to the user and using a user device, the feedback signal according to the feedback parameters.

A second aspect is a device for modifying cognitive processes. The device includes a processor that is configured to receive respective electroencephalogram (EEG) signals from EEG sensors, where the EEG signals are of a brain of a user; extract features from the respective EEG signals; obtain, from a first machine learning (ML) model that uses the features as input, a cognitive state of the brain of the user; obtain, from a second ML model that uses the cognitive state as input, feedback parameters of a feedback signal; and provide, to the user, the feedback signal according to the feedback parameters.

In third aspect is a system for adaptive adjustment of feedback signals are provided. The system includes an acquisition module configured to acquire EEG signals of a user; an extraction module configured to extract features from the EEG signals; a first ML module to obtain a cognitive state of the brain of the user; a second ML module to obtain feedback parameters of a feedback signal based on the cognitive state of the brain of the user; and a feedback module configured to provide a feedback signal to the user according to the feedback parameters.

### BRIEF DESCRIPTION OF DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a diagram of an example of an environment for adaptive user feedback for interfacing with cognitive processes according to implementations of this disclosure.

FIG. 2 is an example of a diagram of a system for modifying cognitive processes according to implementations of this disclosure.

FIG. 3 is a flowchart of an example of a technique for modifying cognitive processes according to implementations of this disclosure.

FIG. 4 depicts an illustrative implementation of a computing system as described herein.

FIGS. 5A-5B are block diagrams of examples of convolutional neural networks (CNNs).

### DETAILED DESCRIPTION

Enhancement or optimization of a cognitive process can be accomplished either through (a) endogenous regulation, whereby the brain learns to use any feedback signal from a point of interaction that contains no internal representation of a brain state, or (b) point of interaction regulation, whereby the point of interaction adjusts its feedback signal based on deviation of brain signals from a set point.

A point of interaction, as used herein, can be defined as some mechanism or device that can provide a feedback signal to a person (also referred to herein as a user) such that the feedback signal is intended to induce a change to the user's brain model and/or activity. The point of interaction can be a hand-held device (e.g., a portable device, etc.), a wearable device (a wrist band, an earbud, a smart watch, etc.), an implantable device, an ambient device, some other device that can be used to provide one or more stimuli to the person, or a combination thereof.

Cognitive processes may include attention, memory, perception, language, and other similar processes. Those may be thought of as primary classes of cognitive processes. In each one of the classes, sub classifications may be further developed. For example, the attention state may include endogenous attention (e.g., attention of a person about what is going on inside the person's body); and exogenous attention (e.g., attention to what is going on in the environment). The attention state may further include memory attention, perception, motor control, and so on. The brain relays (e.g., the brain dynamics underlying those phenomena) may constitute (e.g., considered to be, etc.) a cognitive process.

In an example, a user's brain state of interest may be divided into “focus” versus “mind-wandering.” The dichotomy may be defined as: is the user focused on what the user is doing in the present moment (e.g., having a conversation, writing a report, writing a computer program, studying) versus mind-wandering (e.g., not focused on a task at hand). A wandering mind may be considered to be in a different spatial and/or temporal context. For an example, while studying, the user might start thinking about the vacation that the user is going to have a month from now or the user could be thinking about that conversation the user had with a friend two days ago. Those events can be classified as mind-wandering events because the user is thinking about things that are not in the immediate environment and/or are not a current task. A focused state may be defined as thinking about (e.g., focusing on, etc.) things that the user is currently doing and are currently in the user's immediate environment.

A particular cognitive process (or brain state) can be modified through external feedback. To illustrate, and without loss of generality, as is known, alpha brain waves may be associated with relative calm and relaxation. When, based on EEG signal analysis, it is determined that the brain is producing little Alpha waves, the brain may be considered to not be relaxed. That is, the brain may be considered to be restless or wandering. Thus, to move a wandering or restless mind of a person to a calm and relaxed state, the brain can be induced, such as via some relaxation-inducing feedback signal that is provided to (e.g., output to, delivered to, presented to, etc.) the user, to produce more Alpha waves. In an example, the feedback signal may be an audible signal (e.g., ocean waves sounds, rainforest, etc.) or a visual signal (images of warm colors, calm waters, or the like).

Disclosed herein are implementations of a method for adaptive adjustment of feedback signals. The method includes receiving respective electroencephalogram (EEG) signals of a user; extracting features from the respective EEG signals; obtaining, from a first model that uses the features as input, a cognitive state of the brain of the user; obtaining, from a second model that uses the cognitive state as input, feedback parameters of a feedback signal; and providing, to the user and using a user device, the feedback signal according to the feedback parameters.

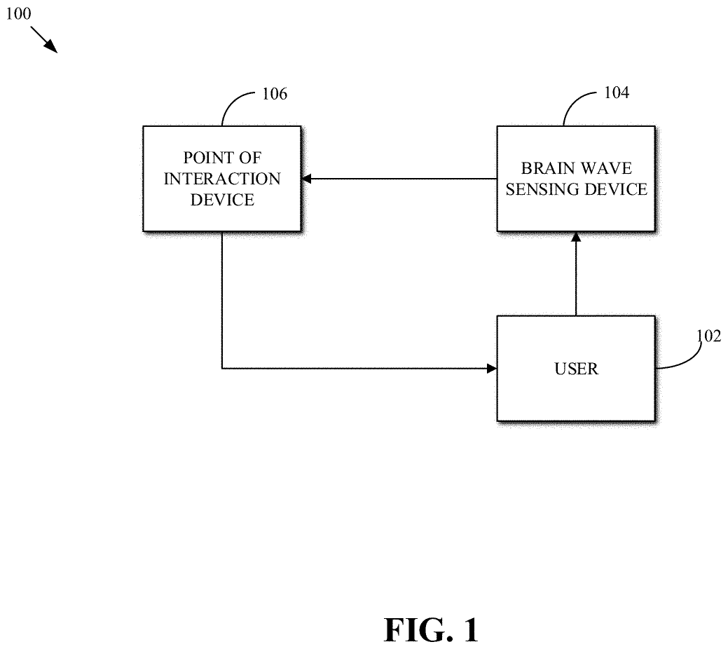

FIG. 1 is a diagram of an example of an environment 100 for adaptive user feedback for interfacing with cognitive processes according to implementations of this disclosure. The environment 100 includes a user 102, a brain-wave-sensing device 104, and a point-of-interaction device 106. The brain-wave-sensing device 104 may be used to acquire EEG signals of the user 102. Based on a determination that a cognitive state of the brain of the user being different from a desired state, the point-of-interaction device 106 may be used to provide an adaptive feedback signal to the user to induce the state of the brain into or toward the desired state. In an example, the cognitive state can be determined by the brain-wave sensing device 104, by the point-on-interaction device 106, by another device not shown in FIG. 1 but that receives the EEG signals, or a combination thereof.

In an example, the brain-wave-sensing device 104 includes sensors (EEG sensors) that may be worn by a user or are in contact with (e.g., affixed to, etc.) the user's head at different locations (e.g., at the forehead, the temples, etc.). The brain-wave-sensing device 104 may be an implantable device or a head-wearable device. For example, the brain-wave-sensing device 104 may be a head-band that the user wears over forehead or around the head. The EEG sensors can be used to obtain (e.g., measure, record, etc.) brain waves (e.g., signals), such as in different parts of the brain. In one aspect, the brain-wave-sensing device may extract out the brain signals. In another aspect, another apparatus may extract the brain signals. The brain waves can include one or more of delta waves, theta waves, alpha waves, beta waves, and/or gamma waves, or other oscillatory signals detectable by the brain-wave-sensing device. A brain state can be defined (e.g., characterized, identified etc.) by the different brain waves and/or signals produced in different locations of the brain.

The point-of-interaction device 106 can be used to provide feedback to the user. The feedback can be visual, audible, haptic, of other modalities, or a combination thereof. In an example, the point-of-interaction device 106 can be a portable device (e.g., a smartphone, etc.). To illustrate, and without loss of generality, a first visual feedback signal that is a blooming flower and a second visual feedback signal that is a contracting flower may be displayed on the portable device. The brain can use that signal as a feedback signal. In an example, the point-of-interaction device 106 can be a wearable device (e.g., earbuds) that includes sensors. In an example, the point-of-interaction device 106 can be a wrist-wearable device and the feedback signal can be provided in the form of a haptic signal whereby different number of taps or vibrations may constitute different feedback signals. In an example, the wrist-wearable device can include a display for displaying visual feedback signals. In an example, the wrist-wearable device can include a speaker for outputting audible feedback signals. In some aspects, a combination of the earbuds, the smartwatch, the phone, the wrist-worn device, and/or some other point of interaction device can produce the feedback signal. In an aspect, the feedback device may be separate from the measurement device.

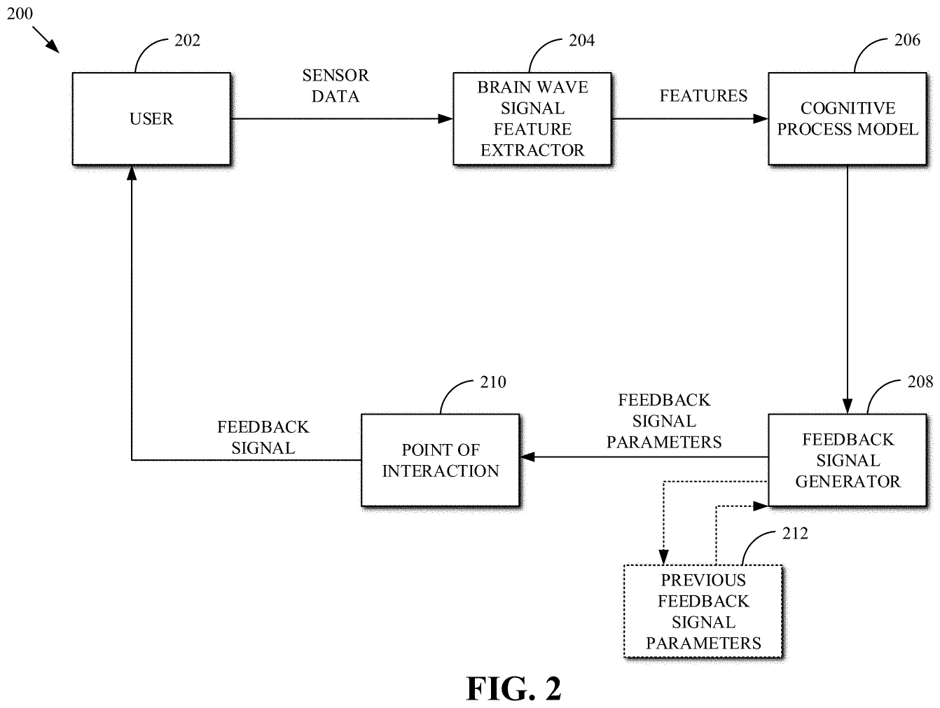

FIG. 2 is an example of a diagram of a system 200 for modifying cognitive processes according to implementations of this disclosure. In one aspect, brain activities in a user 202 over a period of time are measured. The measured brain activities can be parameterized into an information space (e.g., a latent information space) that delineates (e.g., captures the essence of, etc.) the cognitive process occurring in the brain. The information space can represent an estimate of the level of the brain state of the user 202. The information space can be thought of as representing the effectiveness and/or category of the cognitive process of the user 202.

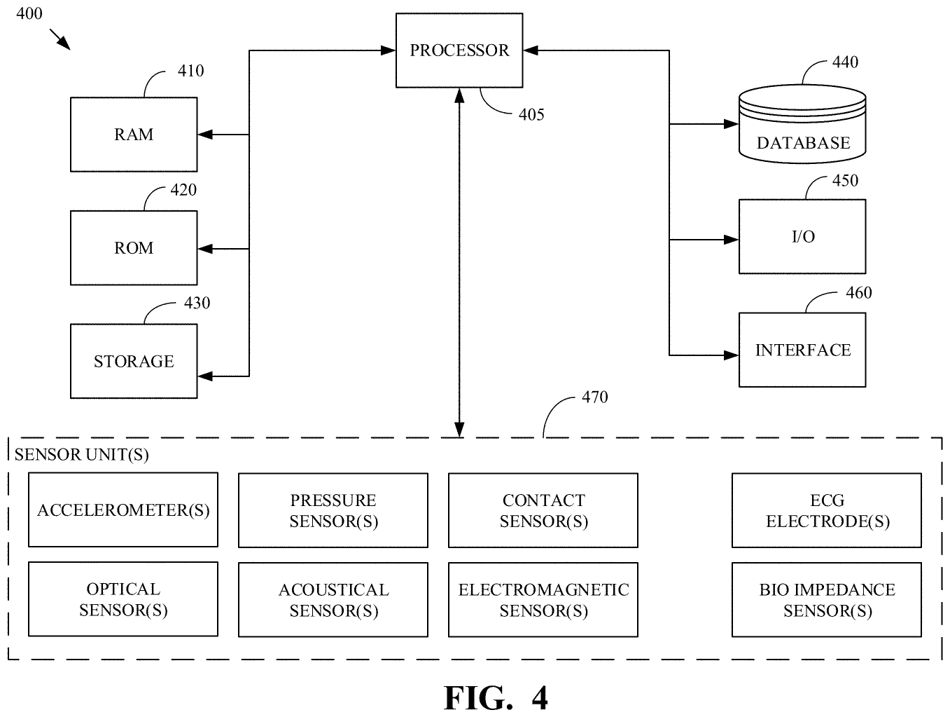

This cognitive process representation may be provided by a cognitive process model module, which is referred to generically herein as a first model 206. The first model 206 can be a machine learning (ML) model (e.g., an output of a ML model), a heuristic graph, some other type of model, or a combination thereof. The first model 206 can represent the cognitive process as a dynamic system rather than a set point. “Set point,” as used herein, can mean a pre-conceived (e.g., fixed, etc.) view of a brain model of a certain desired brain state or brain activities at a time point. The brain signals of the user 202 can be measured by EEG sensors as described above. The brain signals of the user 202 can be measured by a brain-wave sensing device (not shown in FIG. 2), such as the brain-wave-sensing device 104 of FIG. 4. Features can be extracted from the sensor data of the EEG sensors by a feature extractor 204. The features can be input to the first model 206 to obtain the information space of the brain activities. The features can either be explicitly calculated for the model (e.g. the feature extractor 204 is independent from the first model 206), or the features can be intrinsically learned by the first model 206 (e.g. the feature extractor 204 is an integral part of the first model 206, for an example, a convolutional neural network that learns to extract wavelet characteristics to determine the brain state). An example of explicitly calculated features can be hand-crafted features, which refers to, for example, specific computer instructions that isolate the features.

In an example, the feature extractor 204 can use a variety of techniques to extract the features from EEG signals. Such techniques can include one or more of time frequency distributions (TFD), Fast Fourier Transform (FFT), eigenvector methods (EM), wavelet transform (WT), and auto regressive method (ARM), parameterization of the neural power spectrum such as the 1/f slope, relative proportions/power of various frequency bands such as alpha and delta bands, and so on. These features may be learned implicitly by a model, such as a convolutional neural network, in which the information extraction methods and signal transformations are iteratively learned during the model training process.

The information space of the brain activities (i.e., the output of the first model 206) of the user 202 can be provided to a feedback signal generator, which is referred to herein generically as a second model 208. The information space of the brain activities can be provided to the second model 208 at a time point where a feedback signal is to be sent (e.g., provided, displayed, output, etc.) to the user (such as via a point-of-interaction device 210), whereby through ML and/or heuristics, the feedback signal can be adjusted in order to optimize the estimate of the level of effectiveness of the cognitive process.

The adjusted (e.g., adaptive) feedback signal at the point-of-interaction device 210 can be provided to the user using one or more modalities (e.g. visual, auditory, somatic, affective, haptic, etc.), whereby the user is able to respond to the feedback signal by adjusting the user's cognitive states towards a desired state.

In an aspect, the feedback signal may be electrical. For an example, the feedback signal may be a direct current or an alternating current signal. For an example, the electrical signal may be applied on the scalp of a user's head or be applied directly into the cortex of the brain. In an example, an electrical feedback signal may resemble neural activity and/or environmental information. In an aspect, the electrical feedback signal may be applied to stimulate auditory cortex and/or visual cortex. In an aspect, the system may modulate the cortical state of a user through different sensory areas of the brain.

In an aspect, two separate ML models may be used. The first model can form a representation of the brain dynamics using features extracted from EEG signals by the feature extractor 204; and the second model 208 can use the representation of the brain dynamics to output feedback signal parameters. At least one of the first model 206 or the second model 208 may be a convolutional neural network. In an example, the first model 206 and the second model 208 may be combined into one model. In an example, the second model can be a recurrent neural network, which can internally use previous feedback signal parameters to output new feedback signal parameters.

In another example, there may be two inputs for the second model 208. The two inputs may be parameters of one or more previous feedback signals 212 and parameters of the cognitive state or representation of the user brain, which is output by the first model 206. The output of the second model 208 may be the ideal feedback signal based on the brain state and the type, the quality, and/or the parameters of the feedback signal and that tends to move brain toward the desired state. To illustrate, and without loss of generality, given a current brain state and previous feedback signals, slight variation in volume of an audible feedback signal (e.g., fluctuating the volume at a certain frequency) may be determined to be the optimal feedback signal, rather than merely increasing or decreasing volume of the audio signal.

In an aspect, the second model 208 can consider what the brain is doing and what the current state of the feedback signal is, and the second model 208 can then (implicitly) solve this minimization problem, for an example, to learn to solve this responsiveness of the brain to the parameters of the signal.

In one example, the second model 208 may take the feedback signal parameters (e.g., temporal information, characteristics of the wave forms et al.), and at the same time, monitor the response of the brain to that signal. The second model 208 can analyze the brain states after the feedback signals and align the signal parameters with the brain states. In one example, the second model 208 may change the parameters or the temporal structure of the feedback signal in a way that maximizes the brain's response to the adjusted feedback signal.

In an aspect, adjustment of parameters of a feedback signal is relative to the response of the brain to that signal. To illustrate, even in the simplest case of adjusting volume for an audio signal, an instantaneous increase in volume may not be the most ideal feedback signal. Rather, it may be that a lag of time, e.g. three seconds, before increasing the volume may be the optimal feedback signal to get the brain to respond in the desired way.

To reiterate, an information space describing (e.g., codifying, modeling, inferring, classifying, etc.) the brain state of the user 202 can be extracted, by the first model 206, from EEG signals and/or from features extracted from the EEG signals. The extracted information space can depict a picture of the current brain state. A desired state of the user's brain activity may be preconfigured/predetermined/set. An initial feedback signal that attempts to move the current state of the brain to the desired state is provided to the user 202 via the point-of-interaction device 210. More brain states are extracted in a subsequent time window. The new brain state and the desired state are compared, such as implicitly compared by the second model 208. Based on a determination that the brain state is increasingly similar to the desired state, the feedback signal may be modified to a first feedback signal or no new feedback signal is provided to the user 202. In an example, the first feedback signal may be a decrease of the initial feedback signal. Based on a determination that the brain state is increasingly different from the desired state, the feedback signal may be modified to a second feedback signal. In an example, the second feedback signal may be adjusted from the initial feedback signal. In another example, the second feedback signal may be independent from (e.g., unrelated to, not an adjustment to, etc.) the initial feedback signal.

Implementations according to this disclosure allow for adaptive adjustment of feedback signals based on an internal representation of a cognitive process, and an estimate of the effectiveness of that cognitive process.

The feedback signal can be adapted (e.g., parametrized, varied, etc.) in different ways. For example, with respect to an auditory feedback signal, there may be different ways to characterize an auditory feedback signal, such as via pitch, phase of the signal, intensity, more, fewer, other characteristics, or a combination thereof. As such, the feedback signal can be parameterized, such as by the second model 208, by setting (e.g., outputting) different values for at least some of the characteristics (e.g., parameters, variables, etc.) of the feedback signal. In an aspect, an algorithm can dynamically change the feedback signal in a way that maximally pushes the brain into a desired state.

As already mentioned, the first model 206 can model (e.g., parametrize, etc.) the dynamic activity of the brain when certain cognitive process (e.g. relaxation, focus, etc.) is present. In an aspect, rather than having just one snapshot of the brain activity, (e.g., measuring only an amount of alpha waves), the first model 206 may be applied to model the dynamic activities of the brain, such as both the spatial variances and the temporal variances in cognitive process.

For an example, a relaxation state of a brain may not be accurately described by one factor, such as the presence of or the amount of alpha waves. Instead, a more accurate representation of the relaxation state may include several factors that dynamically interact and change over time, such as the different types of brain waves produced in different parts of the brain over time.

In implementations of the present disclosure, algorithms (e.g., inferences, calculations, etc.), such as by the first model 206, can be applied to analyze brain signals (e.g., EEG signals) at different electrodes, and to analyze changes in those signals over time (e.g. 30 seconds, 5 seconds, or 1 second, or some other time intervals or windows). The algorithms can represent changes in brain states or a current brain state measure during a measurement period or time window. For an example, relaxation state may be high Alpha followed by low Alpha followed by high Theta, followed by low theta; additionally, a relaxed state may have different characteristics of brain wave activity on different sensors on the recording device, such as the brain-wave-sensing device 104 of FIG. 1.

In one aspect, algorithms (e.g., inferences, calculations, etc.), such as by the second model 208, can be applied to analyze and adjust (e.g., vary, etc.) feedback signals. For an example, the parameters of the feedback signal, which may be output by the second model 208, can be varied in a way that can maximize the change in brain state towards the desired state, based on one or more previous feedback signals generated according to the parameters output by the ML algorithm. For an example, with respect to an auditory feedback signal, in addition to increasing or decreasing the pitch or the tone of the auditory signal, the sparsity of the signal may also be varied. The second model 208 can monitor changes in the feedback signal and changes in the brain states and synchronize those aspects as much as possible. For an example, previous parameters of the feedback signal and the induced brain state may be used by the ML algorithms to select (e.g., output, calculate, determine, look up, etc.) new parameters for a next feedback signal.

For another example, in order to induce the brain into a desired state, visual signals may be used. Instead of merely a simplistic scheme (such as a green-colored signal indicating that the brain is in the desired state and a red-colored signal indicating that the brain in not in the desired state), different colors may be mixed in. In an aspect, different portions of a visual feedback signal to be displayed on a screen of the point-of-interaction device 210 may be in different colors and/or contrasts. In one implementation, the second model 208 can be trained to learn how to vary the color, the intensity, the pattern, and/or other characteristics of the pixels of a visual feedback signal in a way that maximally induces the brain toward the desired state.

In an example of collecting training data (e.g., ground truth data) for training the first model 206, to induce test persons into a relaxed state from a stressed or wandering state, the test persons may be put into a focus state. For an example, a test person may then be asked to count his/her breaths. Over time, a person's mind starts to wander. The person may realize that his/her mind is wandering and the person starts counting breaths again. Dynamic brain activity signals of that person's brain while the person is focused on counting breaths and while the person's mind is wandering or the person's not paying attention to what he/she is supposed to be doing are recorded and measured. The dynamics of the brain in those states are modeled based on the signals extracted from those dynamic brain activity signals recorded and measured. The data is fed into the ML models during the training phase so that the ML models can learn the dynamics of the brain.

As mentioned above, the cognitive process can be represented as a dynamic system rather than a set point. To provide a simple, fictitious illustration, assume that brain states can be numbered on a scale of 1 to 10 where the desired brain state is 6, which may represent relaxation. In the example, the brain state may be currently at a 1. However, to arrive at 6, more than one stimulus may be necessary to get the brain to the state of 6. In an example, the state 1 may be connected to the state 3, the state 3 may be connected to the state 9, and the state 9 may be connected to the desired state 6. Therefore, rather than just one single value of what the brain should look like, multiple values are allowed to interact with each other and the system may get to that desired 6 in many different ways.

As such, getting the brain to the desired state of 6 may be more than merely setting a set point of 6. Getting the brain to 6 is more of a network of intermediate states than a single set point. The network itself may be a computational graph. In order to achieve the desired state 6, the brain may be moved (via feedback signals) can be by adding 3 to 3, or adding 1 to 5, or by subtracting three from nine. There are many different routes to a 6. Changes over time are contextual and may vary for different users. For an example, the link allowing subtracting 3 from 9 doesn't exist that day. For another example, what construes as the desired state of 6 (e.g. a focused state) may be different between different users. In this network-based and dynamic representation, an available route may be generated get to that desired point.

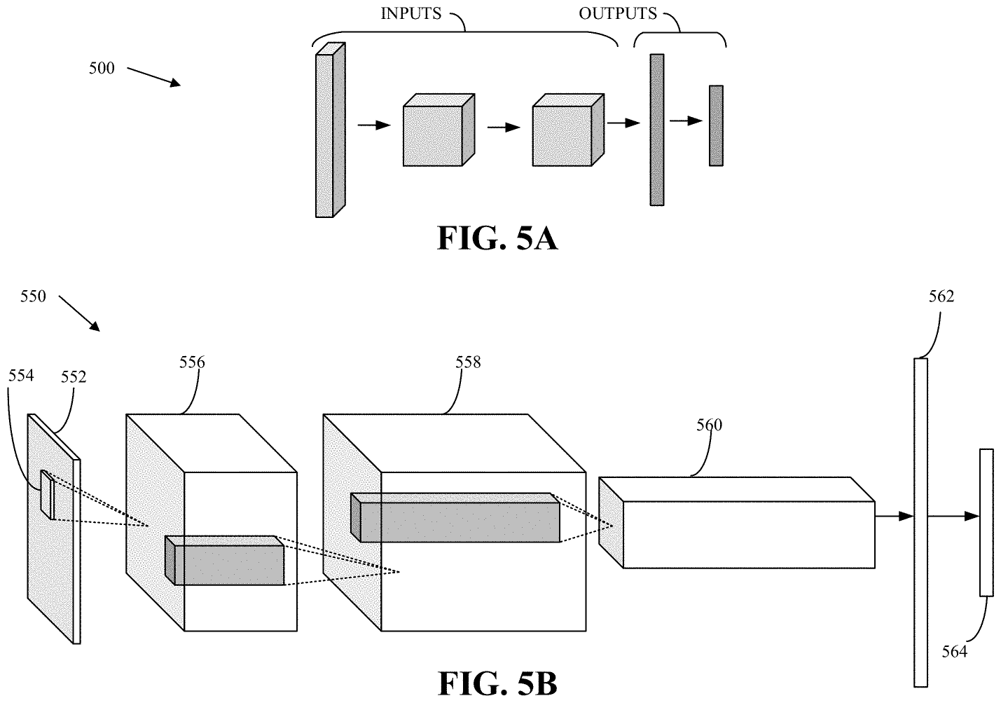

The ML algorithms described herein can be trained by observing and codifying how peoples brain states change. Typical configurations of ML models are described herein with respect to FIGS. 5A-5B.

In an aspect, ML is leveraged to understand the relationship between providing the different parameters of the feedback signal and the brain responding to it. In an aspect, the whole system is not separate, but is continuous.

In an aspect, the ML models may have binary output. For an example, the output of the first model 206 may be mind-wandering (e.g., output value 0) versus focus (e.g., output value 1). In another aspect, the ML models in the system may have multi-label outputs. For an example, the output of the first model 206 may be a weighted exogenous focus, a weighted endogenous focus, a weighted mind-wandering, a weighted concentration parameter, a weighted stress parameter, and so on. In another example, the output may include outputs that answer questions such as: what is the cognitive load? What is the user's emotional state? is the user aroused versus not aroused? is the user focused versus not focused? and so on.

In an aspect, the first model 206 can learn a latent representation of the cognitive process. The outputs of the first model 206 can vary based on what the ML model determines the brain is actually doing and what state it is in.

In an aspect, based on this picture of what the brain is like (e.g., what state the brain is in, what the brain is doing, etc.), parameters of a feedback signal may be varied in a way that moves that model into alignment with the desired brain state. In another aspect, the output of the ML model would be the values of the parameters that are preconfigured to adjust.

In an example, ML algorithms, such as decision trees, a gradient boosting algorithm may be used to determine the interaction of the different aspects of the feedback signal that have a maximal effect on the brain. For an example, to determine whether a user is in a particular state such as a focused state, brain activity signals of the user may be decomposed into differential activities in for example anterior dorsal ventral attentional networks.

In implementation, an apparatus measures brain activities in a user over a period of time (such as a sampling window) is provided. The apparatus parameterizes the brain activities into an information space that delineates the cognitive process occurring, and represents an estimate of the level of effectiveness of a feedback signal in inducing the brain state to move toward a desired state and that can itself be used, as further described below, to further adapt the feedback signal.

The apparatus may provide the information space of the brain activities of the user to a second model, which can determine a time point when a feedback signal is to be sent to the user (the point of interaction), whereby through ML or heuristics, the feedback signal can be adjusted (e.g., adapted, etc.) to induce the brain to move toward a desired state.

The apparatus provides the adjusted feedback signal at the point of interaction to the user through some modality (e.g. visual, auditory, somatic, affective, haptic, etc.), whereby the agent is able to update its internal process.

In yet another implementation, an apparatus measures brain activities in a user over a period of time (e.g., a sampling window). The apparatus may transmit the measured brain activities to a terminal, which parameterizes the brain activities into an information space that delineates the cognitive process occurring, and represents an estimate of the level of effectiveness of the cognitive process. This representation may be provided by a first model, which can be a ML model, a heuristic graph, or some other types of model. The first model can represent the cognitive process as a dynamic system rather than a set point.

In an aspect, an adaptive user interaction system is provided. The system may implement the method and/or apparatus described herein. The system may include two ML models. The system may also include a Brain-Computer Interface (BCI). The BCI may capture and/or measure brain activity signals. A first model can receive the brain activity signals from the BCI as an input. The brain activity signals may be spatial distributions, time varying signal or the like. A second model may perform as a controller system for feedback signals. The second model may take representation of the brain state determined by the first model as one of the inputs. The second model may also take the parameters of one or more previous feedback signals 212 from the last step as one of the inputs. As such, the second model 208 can used the parameters of the one or more previous feedback signals 212 and the latent representation of the current brain state to infer the impact of previous signals on the brain state and provide parameters for a next feedback signal.

In an aspect, BCI may be non-invasive or invasive. For an invasive BCI, it may involve opening up the scalp, putting electrodes directly onto the cortex or within the cortex. For a non-invasive BCI, it may have sensors just on the scalp. In another aspect, non-invasive BCI may be combined with invasive BCI. In an aspect, one or more sensors may be placed across the forehead. In another aspect, one or more sensors may be placed in and around the hairline behind the ears. In yet another aspect, one or more sensors may be distributed on a user's head using nasion, inion, and/or preauricular points on left and right ears as reference points. In yet another aspect, one or more sensors may be distributed on a user's head using the main cortices of human brain as reference points.

In an aspect, the sensors may be electroencephalography (EEG) sensors. In another aspect, the sensors may be voltage sensors. In yet another aspect, the sensors may be optical sensors. In yet another aspect, the sensors may be imaging sensor.

In an aspect, the brain dynamics of the cognitive representation are based on measurements from all sensors across the brain. For an example, an apparatus may have four sensors, with 2 placed on forehead and 2 behind ears. In one example, values for Alpha waves across all four sensors may be averaged. In another example, values for Alpha waves across all four sensors may be weighted average. In an aspect, certain electrodes are more sensitive to dynamics and subcomponents of the neural networks in the brain. In that aspect, merely looking at more or less across all of the sensor is not sufficient to capture the true representation of cognitive processes.

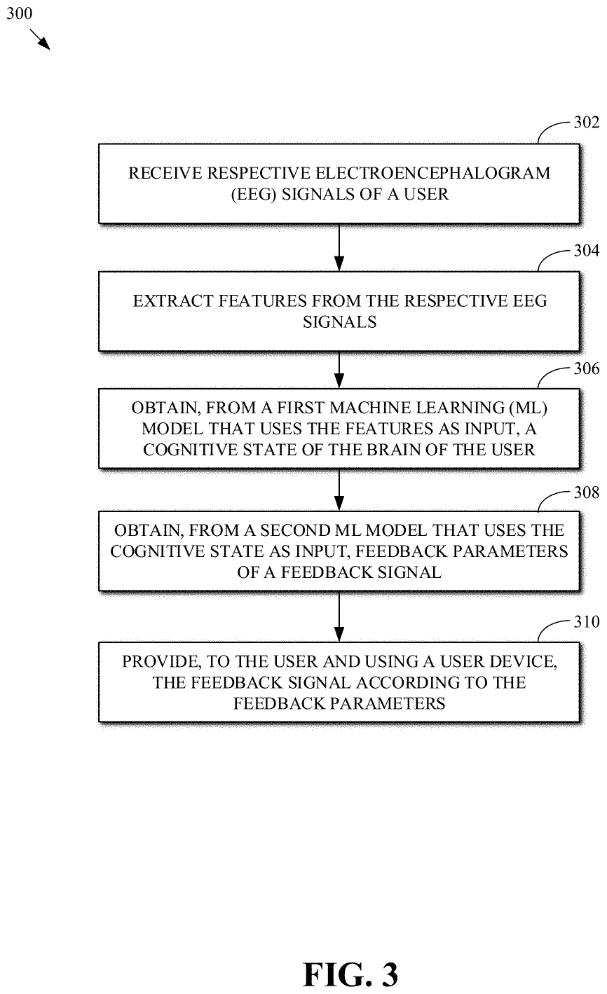

FIG. 3 is a flowchart of an example of a technique 300 for modifying cognitive processes according to implementations of this disclosure. In one example, the technique 300 may be implemented by a system such as the system 200 of FIG. 2.

At 302, the technique 300 receive respective electroencephalogram (EEG) signals of a user. The respective EEG signals are received from EEG sensors and are signals of the brain of the user. The respective EEG signals can be acquired by the brain-wave-sensing device 104 as described above. The respective EEG signals can be one or more of the sensor data as described above with respect to FIGS. 1 and 2.

At 304, the technique 300 extracts features from the respective EEG signals. The extraction of the features may be performed by the brain-wave-sensing device 104 or the feature extractor 204 as described above. The features extracted can be one or more of the brain wave features as described above with respect to FIGS. 1 and 2.

At 306, the technique 300 obtains, from a first machine learning (ML) model that uses the features as input, a cognitive state of the brain of the user. The first model can be the first model 206 of FIG. 2 as described above.

At 308, the technique 300 obtains, from a second model that uses the cognitive state as input, feedback parameters of a feedback signal. The feedback signal parameters may be generated by the second model 208 of FIG. 2 as described above.

At 310, the technique 300 provides, to the user and using a user device, the feedback signal according to the feedback parameters. The feedback signal may be output by the point of interaction device of FIGS. 1 and 2 as described above. The user device can be one or more of the point of interaction device 106 of FIG. 1 or modalities at the point-of-interaction device 210 of FIG. 2 as described above.

In an example, the cognitive state of the brain of the user can include a classification of whether the brain is focused or is wandering. In an example, the cognitive state of the brain of the user can include a weighted exogenesis focus, a weighted endogenous focus, a weighted mind-wandering, a weighted concentration parameter, and a weighted stress parameter. In an example, extracting the features from the respective EEG signals includes extracting the features from the respective EEG signals by a feature extractor and the feature extractor is separate from the first ML model. In an example, extracting the features from the respective EEG signals includes extracting the features from the respective EEG signals by the first ML model.

In an example, the technique obtains the feedback signal parameters. In an example, the feedback signal parameters can be obtained from the second model 208 as described above. The second model further uses previous parameters of the feedback signal as one of the inputs. In an example, the wearable device is a wrist-worn device and the feedback signal is a haptic feedback signal. In another example, the wearable device is a portable device that outputs audio and the feedback signal is an audio feedback signal. In an example, the feedback parameters can include at least two of a pitch, tone, duration, and a delay of the audio feedback signal.

FIG. 4 depicts an illustrative processor-based computing system (i.e., a system 400) representative of the type of computing system that may be present in or used in conjunction with any aspect of point-of-interaction device 106 and/or the brain-wave-sensing device 104 of FIG. 1 comprising electronic circuitry according to implementations of this disclosure, wherein each may comprise any one or more components of system 400.

The system 400 may be used in conjunction with any one or more of transmitting signals to and from the one or more accelerometers, sensing or detecting signals received by one or more sensors of sensing device 104, processing received signals from one or more components or sensors of sensing device 104 or a secondary device, and storing, transmitting, or displaying information. The system 400 is illustrative only and does not exclude the possibility of another processor- or controller-based system being used in or with any of the aforementioned aspects of sensing device 104.

In one aspect, system 400 may include one or more hardware and/or software components configured to execute software programs, such as software for storing, processing, and analyzing data. For example, system 400 may include one or more hardware components such as, for example, processor 405, a random access memory module (RAM) 410, a read-only memory module (ROM) 420, a storage system 430, a database 440, one or more input/output (I/O) modules 450, an interface module 460, and one or more sensor modules 470. Alternatively and/or additionally, system 400 may include one or more software components such as, for example, a computer-readable medium including computer-executable instructions for performing methods consistent with certain disclosed implementations. It is contemplated that one or more of the hardware components listed above may be implemented using software. For example, the storage system 430 may include a software partition associated with one or more other hardware components of system 400. System 400 may include additional, fewer, and/or different components than those listed above. It is understood that the components listed above are illustrative only and not intended to be limiting or exclude suitable alternatives or additional components.

Processor 405 may include one or more processors, each configured to execute instructions and process data to perform one or more functions associated with system 400. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 4, processor 405 may be communicatively coupled to RAM 410, ROM 420, the storage system 430, database 440, I/O module 450, interface module 460, and one more of the sensor modules 470. Processor 405 may be configured to execute sequences of computer program instructions to perform various processes, which will be described in detail below. The computer program instructions may be loaded into RAM for execution by processor 405.

RAM 410 and ROM 420 may each include one or more devices for storing information associated with an operation of system 400 and/or processor 405. For example, ROM 420 may include a memory device configured to access and store information associated with system 400, including information for identifying, initializing, and monitoring the operation of one or more components and subsystems of system 400. RAM 410 may include a memory device for storing data associated with one or more operations of processor 405. For example, ROM 420 may load instructions into RAM 410 for execution by processor 405.

The storage system 430 may include any type of storage device configured to store information that processor 405 may need to perform processes consistent with the disclosed implementations.

Database 440 may include one or more software and/or hardware components that cooperate to store, organize, sort, filter, and/or arrange data used by system 400 and/or processor 405. For example, database 440 may include user profile information, historical activity and user-specific information, physiological parameter information, predetermined menu/display options, and other user preferences. Alternatively, database 440 may store additional and/or different information.

I/O module 450 may include one or more components configured to communicate information with a user associated with system 400. For example, I/O module 450 may comprise one or more buttons, switches, or touchscreens to allow a user to input parameters associated with system 400. I/O module 450 may also include a display including a graphical user interface (GUI) and/or one or more light sources for outputting information to the user. I/O module 450 may also include one or more communication channels for connecting system 400 to one or more secondary or peripheral devices such as, for example, a desktop computer, a laptop, a tablet, a smart phone, a flash drive, or a printer, to allow a user to input data to or output data from system 400.

The interface module 460 may include one or more components configured to transmit and receive data via a communication network, such as the Internet, a local area network, a workstation peer-to-peer network, a direct link network, a wireless network, or any other suitable communication channel. For example, the interface module 460 may include one or more modulators, demodulators, multiplexers, demultiplexers, network communication devices, wireless devices, antennas, modems, and any other type of device configured to enable data communication via a communication network.

System 400 may further comprise one or more sensor modules 470. In one implementation, sensor modules 470 may comprise one or more of an accelerometer module, an optical sensor module, and/or an ambient light sensor module. Of course, these sensors are only illustrative of a few possibilities and sensor modules 470 may comprise alternative or additional sensor modules suitable for use in sensing device 104. It should be noted that although one or more sensor modules are described collectively as sensor modules 470, any one or more sensors or sensor modules within sensing device 104 may operate independently of any one or more other sensors or sensor modules. Moreover, in addition to collecting, transmitting, and receiving signals or information to and from sensor modules 470 at processor 405, any one or more sensors of sensor module 470 may be configured to collect, transmit, or receive signals or information to and from other components or modules of system 400, including but not limited to database 440, I/O module 450, or the interface module 460.

FIGS. 5A-5B are block diagrams of examples 500 and 550 of convolutional neural networks (CNNs) for mode decisions.

FIG. 5A illustrates a high level block diagram of an example 500 of a typical CNN network, or simply a CNN. As mentioned above, a CNN is an example of a machine-learning model. In a CNN, a feature extraction portion typically includes a set of convolutional operations, which is typically a series of filters that are used to filter an input signal based on a filter. For example, and in the context of EEG signal analysis, these filters can be used to find features in EEG signals. The features can be used to map the features to a brain state. As the number of stacked convolutional operations increases, later convolutional operations can find higher-level features.

In a CNN, a classification portion is typically a set of fully connected (FC) layers, which may also be referred to as dense operations. The fully connected layers can be thought of as looking at all the input features of a EEG signals in order to generate a high-level classifier. Several stages (e.g., a series) of high-level classifiers eventually generate the desired classification output.

As mentioned, a typical CNN network is composed of a number of convolutional operations (e.g., the feature-extraction portion) which may be followed by a number of fully connected layers. The number of operations of each type and their respective sizes is typically determined during the training phase of the machine learning. As a person skilled in the art recognizes, additional layers and/or operations can be included in each portion. For example, combinations of Pooling, MaxPooling, Dropout, Activation, Normalization, BatchNormalization, and other operations can be grouped with convolution operations (i.e., in the features-extraction portion) and/or the fully connected operation (i.e., in the classification portion). The fully connected layers may be referred to as Dense operations. As a person skilled in the art recognizes, a convolution operation can use a SeparableConvolution2D or Convolution2D operation.

As used in this disclosure, a convolution layer can be a group of operations starting with a Convolution2D or SeparableConvolution2D operation followed by zero or more operations (e.g., Pooling, Dropout, Activation, Normalization, BatchNormalization, other operations, or a combination thereof), until another convolutional layer, a Dense operation, or the output of the CNN is reached. Similarly, a Dense layer can be a group of operations or layers starting with a Dense operation (i.e., a fully connected layer) followed by zero or more operations (e.g., Pooling, Dropout, Activation, Normalization, BatchNormalization, other operations, or a combination thereof) until another convolution layer, another Dense layer, or the output of the network is reached. The boundary between feature extraction based on convolutional networks and a feature classification using Dense operations can be marked by a Flatten operation, which flattens the multidimensional matrix from the feature extraction into a vector.

In a typical CNN, each of the convolution layers may consist of a set of filters. While a filter is applied to a subset of the input data at a time, the filter is applied across the full input, such as by sweeping over the input. The operations performed by this layer are typically linear/matrix multiplications. The output of the convolution filter may be further filtered using an activation function. The activation function may be a linear function or non-linear function (e.g., a sigmoid function, an arcTan function, a tanH function, a ReLu function, or the like).

Each of the fully connected operations is a linear operation in which every input is connected to every output by a weight. As such, a fully connected layer with N number of inputs and M outputs can have a total of N×M weights. As mentioned above, a Dense operation may be generally followed by a non-linear activation function to generate an output of that layer.

Some CNN network architectures may include several feature extraction portions that extract features at different granularities and a flattening layer (which may be referred to as a concatenation layer) that receives the output(s) of the last convolution layer of each of the extraction portions. The flattening layer aggregates all the features extracted by the different feature extraction portions into one input set. The output of the flattening layer may be fed into (i.e., used as input to) the fully connected layers of the classification portion.

FIG. 5B illustrates a high level block diagram of an example 550 of a CNN. In CNNs such as the example 550, convolutional layers are used for extracting features and fully connected layers are used as the classification layers.

In the example 550, EEG signals 554 of a EEG vector date 552 can be fed through one or more convolutional layers (e.g., convolutional layers 556 and 558), one or more max pooling layers (e.g., a pooling layer 560), and one or more fully connected layers (e.g., fully connected layers 562) to produce an output at an output layer 564. As mentioned above, the output can be a latent space representation of a brain state (i.e., a cognitive process) as described with respect to the first model 206 of FIG. 2.

In another example, the latent space representation of a brain state can be input through the input layer and at the output layer 564, parameters of a next feedback signal are obtained.

While implementations have been illustrated and described, it will be appreciated that various changes can be made therein without departing from the spirit and scope of the disclosure. Moreover, the various features of the implementations described herein are not mutually exclusive. Rather any feature of any implementation described herein may be incorporated into any other suitable implementation.

Additional features may also be incorporated into the described systems and methods to improve their functionality. For example, those skilled in the art will recognize that the disclosure can be practiced with a variety of physiological monitoring devices, including but not limited to heart rate and blood pressure monitors, and that various sensor components may be employed. The devices may or may not comprise one or more features to ensure they are water resistant or waterproof. Some implementations of the devices may hermetically sealed.

Other implementations of the aforementioned systems and methods will be apparent to those skilled in the art from consideration of the specification and practice of this disclosure. It is intended that the specification and the aforementioned examples and implementations be considered as illustrative only, with the true scope and spirit of the disclosure being indicated by the following claims. While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for modifying cognitive processes, comprising:
receiving respective electroencephalogram (EEG) signals from EEG sensors, wherein the EEG signals are of a brain of a user;
extracting features from the respective EEG signals;
obtaining, from a first machine learning (ML) model that uses the features as input, a cognitive state of the brain of the user;
obtaining, from a second ML model that uses the cognitive state as input, feedback parameters of a feedback signal; and
providing, to the user and using a user device, the feedback signal according to the feedback parameters.

2. The method of claim 1, wherein the cognitive state of the brain of the user comprises a classification of whether the brain is focused or is wandering.

3. The method of claim 1, wherein the cognitive state of the brain of the user comprises a weighted exogenesis focus, a weighted endogenous focus, a weighted mind-wandering, a weighted concentration parameter, and a weighted stress parameter.

4. The method of claim 1, wherein extracting the features from the respective EEG signals comprises:
extracting the features from the respective EEG signals by a feature extractor wherein the feature extractor is separate from the first ML model.

5. The method of claim 1, wherein extracting the features from the respective EEG signals comprises:
extracting the features from the respective EEG signals by the first ML model.

6. The method of claim 1, wherein the second ML model further uses previous parameters of the feedback signal as input.

7. The method of claim 1, wherein the user device is a wrist-worn device and the feedback signal is a haptic feedback signal.

8. The method of claim 1, wherein the user device is a portable device that outputs audio and the feedback signal is an audio feedback signal.

9. The method of claim 8, wherein the feedback parameters comprise at least two of a pitch, tone, duration, and a delay of the audio feedback signal.

10. A device for modifying cognitive processes, comprising:
a processor configured to:
receive respective electroencephalogram (EEG) signals from EEG sensors, wherein the EEG signals are of a brain of a user;
extract features from the respective EEG signals;
obtain, from a first machine learning (ML) model that uses the features as input, a cognitive state of the brain of the user;
obtain, from a second ML model that uses the cognitive state as input, feedback parameters of a feedback signal; and
provide, to the user, the feedback signal according to the feedback parameters.

11. The device of claim 10, wherein the cognitive state of the brain of the user comprises a classification of whether the brain is focused or is wandering.

12. The device of claim 10, wherein the cognitive state of the brain of the user comprises a weighted exogenesis focus, a weighted endogenous focus, a weighted mind-wandering, a weighted concentration parameter, and a weighted stress parameter.

13. The device of claim 10, wherein to extract the features from the respective EEG signals comprises to:
extract the features from the respective EEG signals by a feature extractor wherein the feature extractor is separate from the first ML model.

14. The device of claim 10, wherein to extract the features from the respective EEG signals comprises to:
extract the features from the respective EEG signals by the first ML model.

15. The device of claim 10, wherein the second ML model further uses previous parameters of the feedback signal as input.

16. The device of claim 10, wherein the device is a wrist-worn device and the feedback signal is a haptic feedback signal.

17. The device of claim 10, wherein the device is a portable device that outputs audio and the feedback signal is an audio feedback signal.

18. The device of claim 17, wherein the feedback parameters comprise at least two of a pitch, tone, duration, and a delay of the audio feedback signal.

19. A system for adaptive adjustment of feedback signals, comprising:
an acquisition module configured to acquire EEG signals of a user;
an extraction module configured to extract features from the EEG signals;
a first ML module to obtain a cognitive state of a brain of the user;
a second ML module to obtain feedback parameters of a feedback signal based on the cognitive state of the brain of the user; and
a feedback module configured to provide the feedback signal to the user according to the feedback parameters.

20. The system of claim 19, wherein the cognitive state of the brain of the user comprises a weighted exogenesis focus, a weighted endogenous focus, a weighted mind-wandering, a weighted concentration parameter, and a weighted stress parameter.
